SEQUENCE LISTING (21 sequences)

SEQ ID NO: 1 - protein, 125 aa, synthetic construct
QVQLVQSGGGLVQAGGSLRLSCAASGSSSSIIAMGWYRQAPGKQRELVATISNGDTTNYIDSVKGRFTISRDNAKNTVYLQMASLEPEDTAVYYCNARQTKWYAAGYEYRTYDYWGQGTQVTVSS

SEQ ID NO: 2 - protein, 125 aa, synthetic construct
QVQLVQSGGGLVQAGGSLRLSCAASGSSSSIIAMGWYRQAPGKQRELVATISNGDTTNYIDSVKGRFTISRDNAKNTVYLQMASLEPEDTAVYYCNARQTKWYAAGYKYRTYDYWGQGTQVTVSS

SEQ ID NO: 3 - protein, 127 aa, synthetic construct
QVQLVQSGGGLVQAGGSLRLSCAASERTFSTYAMGWFRQIPGKERLFVAAVNWNGKTIRYADSVKGRFTISRDNAKNTIALQMNSLKPEDTAVYYCALRSTPMYFTNLASQESYNYWGPGTQVTVSS

SEQ ID NO: 4 - protein, 125 aa, synthetic construct; identical to SEQ ID NO: 1

SEQ ID NO: 5 - protein, 125 aa, synthetic construct
EVQLVESGGGLVQPGGSLRLSCAASGRIFSISNMGWYRQAPGKQRELVATITSGGSTNYGDSVKGRFTISMANAKNAVYLQMNSLKPEDTAVYYCNAGISRRTGTYSGGRYSDYWAQGTQVTVSS

SEQ ID NO: 6 - protein, 125 aa, Synthetic construct
QVQLVQSGGGLVQPGGSLRLSCAASGRVSEINTMGWYRQAPGKQRELVALITSTSSTMYSDSVKGRFTVSRDVAKNMVYLQMNSLKPEDTAVYYCNARQTKWYAAGYEYRTYDYWGQGTQVTVSS

SEQ ID NO: 7 - protein, 127 aa, Synthetic construct
QVQLQESGGGLVQAGGSLRLSCAASERTFSTYAMGWFRQTPGKERLFVAAVNWNGKTIRYADSVKGRFTISRDNAKNTMSLQMNSLKPEDTAVYYCALRSTPMYFTNLASQESYNYWGPGTQVTVSS

SEQ ID NO: 8 - protein, 125 aa, Synthetic construct
QVQLVQSGGGLVQPGGSLRLSCAASGRIFSISNMGWYRQAPGKQRELVATITSGGSTNYGDSVKGRFTISMVNAKNAVYLQMNSLKPEDTAVYYCNAGISRRTGTYSGGRYSDYWGQGTQVTVSS

SEQ ID NO: 9 - protein, 125 aa, Synthetic construct
QVQLVQSGGGLVQAGGSLRLSCAASGSSSSIIAMGWYRQAPGKQRELVATISNGDTTNYTDSVKGRFTISRDKAKNTVYLQMASLEPEDTAVYYCNARQTKWYAAGYVYRTYDYWGQGTQVTVSS

SEQ ID NO: 10 - protein, 125 aa, Synthetic construct
QVQLVQSGGGLVQPGGSLRLGCAASGRVSEINTMGWYRQAPGKQRGLVALITSTSSTMYSDSVKGRFTVSRDVAKNMVYLQMNSLKPEDTAVYYCNARLTRWYAAGYKYRTYDYWGQGTQVTVSS

SEQ ID NO: 11 - protein, 125 aa, Synthetic construct
QVQLVQSGGGLVQAGGSLRLSCAASGSSSSIIAMGWYRQAPGKQRELVATISNGDTTNYIDSVNGRFTISRDNAKNTVYLQMTSLEPEDTAVYYCHARQTKWYAAGYKYRTYDYWGQGTQVTVSS

SEQ ID NO: 12 - protein, 125 aa, Synthetic construct
QVQLVQSGGGLVQPGGSLRLSCAASGRVSEINTMGWYRLAPGKQRELVALITSTSSTMYSDSVKGRFTVSRDVAKNMVYLQMNSLKPEDTAVYYCNARQTKWYAAGYKYRTYDYWGQGTQVTVSS

SEQ ID NO: 13 - protein, 125 aa, Synthetic construct
QVQLVQSGGGLVQPGGSLRLSCAASGRVSEINTMGWYRQAPGKQRELVALITSTSSTMYSDSVKGRFTVSRDVAKNMVYLQMNSLKPEDTAVYYCNARQTIWYAAGYKYRTYDYWGLGTQVTVSS

SEQ ID NO: 14 - protein, 125 aa, Synthetic construct
QVQLVQSGGGLVQSGGSLRLSCAASGSSSSIIAMGWYRQAPGKQRELVATISNGDTTNYIDSVKGRFTISRDNAKNTVYLQMASLEPEDTAVYYCNARQTKWYAAGYEYRTYDYWGQGTQVTVSS

SEQ ID NO: 15 - protein, 125 aa, Synthetic construct
QVQLVQSGGGLVQAGGSLRLSCAASGSSSSIIAMGWYRQAPGKQRELVATISNNDTTNYIDSVKGRFTISRDNAKNTVYLQMASLEPEDTAVYYCNARQTKWYAAGYKYRTYDYWGQGTQVTVSS

SEQ ID NO: 16 - protein, 125 aa, Synthetic construct
QVQLVQSGGGQVQAGGSLRLSCAASGSSSSIIAMGWYRQAPGKQRELVATISNGDTTNYIDSVKGRFTISRDNAKNTVYLQMASLEPEDTAVYYCNARQTKWYAAGYKYRTYDYWGQGTQVTVSS

SEQ ID NO: 17 - protein, 125 aa, Synthetic construct
QVQLVQSGGGLAQAGGSLRLSCAASGSSSSIIAMGWYRQAPGKQRELVATISNGDTTNYIDSVKGRFTISRDNAKNTVYLQMASLEPEDTAVYYCNARQTKWYAAGYKYRTYDYWGQGTQVTVSS

SEQ ID NO: 18 - protein, 125 aa, Synthetic construct
QVQLVQSGGGLVQPGGSLRLSCAASGRVSEINTMGWFRQAPGKQRELVALITSTSSTMYSDSVKGRFTVSRDVAKNMVYLQMNSLKPEDTAVYYCNARQTKWYAAGYKYRTYDYWGQGTQVTVSS

SEQ ID NO: 19 - protein, 125 aa, Synthetic construct
QVQLVQSGGGLVQAGGSLRLSCAASGSSSSIIAMGWYRQAPGKQRELVATISNGDTTNYIDSVKGRFTISRDNAKNTVYLQMASLEPEDTAVYYCNARQTRWYAAGYKYRTYDYWGQGTQVTVSS

SEQ ID NO: 20 - protein, 125 aa, Synthetic construct
QVQLVQSGGGLVQPGGSLRLSCAASGRVSEINTVGWYRQAPGKQRELVALFTSTSSTMYSDSVKGRFTVSRDVAKNMVYLQMNSLRPEDTAVYYCNARQTKWYAAGYKYRTYDYWGQGTQVTVSS

SEQ ID NO: 21 - protein, 125 aa, Synthetic construct
QVQLVQSGGGLVQAGGSLRLSCAASGSSSSIIAMGWYRQAPGKQRELVATISNGDTTNYIDSVKGRFTISRDNAKNTVYLRMASLEPEDTAVYYCNARQTKWYAAGYKYRTYDYWGQGTQVTVSS

(19) United States
(12) Patent Application Publication
(10) Pub. No.: US 2026/0308272 A1
(43) Pub. Date: Oct. 8, 2026

(54) SLC4A1 (BAND 3) BINDING SINGLE DOMAIN ANTIBODIES FOR INCREASED BLOOD RETENTION

(71) Applicant: The Research Foundation for The State University of New York, Amherst, NY (US)

(72) Inventors: Joseph BALTHASAR, Lancaster, NY (US); Brandon BORDEAU, Elma, NY (US); Toan Duc NGUYEN, Buffalo, NY (US)

(21) Appl. No.: 19/512,624

(22) PCT Filed: Aug. 21, 2024
(86) PCT No.: PCT/US2024/043288

§ 371 Date: Feb. 19, 2026

Related U.S. Application Data
(60) Provisional application No. 63/520,689, filed on Aug. 21, 2023.

(51) Int. Cl. C07K 16/28; A61K 39/00; A61K 47/68

(52) U.S. Cl. CPC C07K 16/2896; A61K 47/68033; A61K 47/6855; A61K 2039/505; C07K 2317/22; C07K 2317/31; C07K 2317/569; C07K 2317/92

(57) ABSTRACT

Provided are binding partners that specifically bind to erythrocyte protein SLC4A1 transporter (Band 3) that is highly expressed on erythrocytes. The binding partners are provided as single-specific, bi-specific or multi-specific agents that have improved half-life and/or are effective at a lower concentration. A second component of a bispecific binding partner can bind to a payload-binding selectivity enhancer (PBSE), such as a drug that has been detached from an anti-body drug conjugate. In non-limiting embodiments the PBSE specifically binds to a drug that was administered to an individual as a component of an ADC.

### CROSS REFERENCE TO RELATED APPLICATION

This application claims the benefit of U.S. Provisional Patent Application No. 63/520,689, filed Aug. 21, 2023, the entire disclosure of which is incorporated herein by reference.

### SEQUENCE LISTING

The instant application contains a Sequence Listing which has been submitted in XML format and is hereby incorporated by reference in its entirety. Said XML copy, created on Aug. 16, 2024, is named “01152001858_ST26.xml”, and is 21,827 bytes in size.

### BACKGROUND OF THE DISCLOSURE

Many biological and pharmaceutical agents exhibit desirable cellular pharmacokinetic and pharmacodynamic attributes (e.g., highly selective binding, high affinity, desirable modulation of targeted signaling pathways) but only limited therapeutic potential due to poor systemic pharmacokinetics (e.g., rapid elimination, short biological half-life). While several half-life extension strategies have been invented and are currently utilized for FDA approved therapeutics including albumin binding, PEGylation and Fc-fusion, additional strategies are needed to enable improved half-life extension and higher blood:tissue ratios. The present disclosure is pertinent to this need.

### SUMMARY OF THE DISCLOSURE

This disclosure provides compositions that exhibit an extended half-life and methods of using the compositions. The compositions include an antibody or antigen binding fragment thereof that specifically binds to erythrocytes, and as such, may be referred to as erythrocyte binding partners (“EBPs” in the plural and “EBP” in the singular). As is known in the art erythrocytes may also be referred to as red blood cells (“RBCs”). The binding domains of the EBPs allow tunable half-life extension through modulation of erythrocyte binding affinity. The EBP binding domains, via specificity for erythrocytes, limit protein distribution beyond the blood space which is desirable for therapeutic proteins that require high blood:tissue ratios. In certain embodiments the EBPs are single chain antibodies or antigen binding fragments thereof.

Among utilities of the EBPs is the ability to impart to them a bi- or multi-functionality by, for example, connecting an EBP to another component that targets, e.g., specifically binds, to an agent that is administered to an individual. Thus, in embodiments, the disclosure provides an EBP as a first component of a multi-component molecule, and wherein a second component is a binding partner that specifically binds to another agent, such as a biologically active compound (i.e., payload) that is administered to an individual. The second component may therefore be referred to as a payload-binding selectivity enhancer (PBSE).

In non-limiting embodiments, the PBSE that is a component of a described multi-component molecule may have therapeutic utility in decreasing undesired off-target toxicity of antibody drug conjugates (ADC), particularly when the drug component of the ADC is liberated from the antibody and thereby causes toxicity to an individual. Thus, in a non-limiting embodiment, the second component, e.g., the second component provided as a PBSE, specifically binds to a drug that was administered to an individual as a component of an ADC. Conjugation of the EBP with the PBSE may enable decreased efficiency of the elimination of the multi-component molecule, thereby increasing its half-life and, potentially, increasing therapeutic utility in decreasing undesired off-target toxicity of ADCs. The disclosure includes administering multi-specific constructs, comprised of at least an EBP and a PBSE, in combination with ADCs to decrease the undesired toxicity of ADCs and to increase the therapeutic selectivity of ADC therapy.

### BRIEF DESCRIPTION OF THE FIGURES

FIG. 1. (A) SDS-PAGE analysis of extracted human band 3 protein from RBC used for immunization and screening. (B) Assessment of the anti-RBC antibodies binding to human and mouse RBC by flow cytometry. Cell culture media from positive clones were analyzed. RA1, RA8 (SEQ ID NO: 3), RB12 (SEQ ID NO: 1), RD1 (SEQ ID NO: 4), RD11 (SEQ ID NO: 5), RE8 (SEQ ID NO: 6), RG10 (SEQ ID NO: 7) and RH5 (SEQ ID NO: 8) are anti-human-RBC antibodies that were identified initially through screening.

FIG. 2. Binding assessment via flow cytometric analysis of purified RB12 (SEQ ID NO: 1) and RE8 (SEQ ID NO: 6) to human and mouse red blood cells (A), (B). RB12 (SEQ ID NO: 1) and RE8 (SEQ ID NO: 6) are anti-RBC antibodies that showed cross-reactivity to mouse RBC.

FIG. 3. Pharmacokinetics (PK) study with the anti-RBC sdAbs in mouse. RB12 (SEQ ID NO: 1) and RE8 (SEQ ID NO: 6) are the anti-RBC antibodies. 1HE is a control non-binding antibody.

FIG. 4. Sequence alignments of the parent and derivative anti-RBC clones. CDRs appear in boxes. The top panel shows sequences of initially identified anti-RBC antibodies RA8 (SEQ ID NO: 3), RG10 (SEQ ID NO: 7), RD11 (SEQ ID NO: 5), RH5 (SEQ ID NO: 8), RB12 (SEQ ID NO: 1), RE8 (SEQ ID NO: 6). The bottom panel shows sequences of affinity-matured antibodies to mouse RBC RMA1 (SEQ ID NO: 9), RME2 (SEQ ID NO: 14), RMF1 (SEQ ID NO: 11), RMG3 (SEQ ID NO: 21), RMD3 (SEQ ID NO: 19), RMH2 (SEQ ID NO: 17), RMG2 (SEQ ID NO: 16), RMC1 (SEQ ID NO: 2), RMF2 (SEQ ID NO: 15), RMD1 (SEQ ID NO: 10), RME3 (SEQ ID NO: 20), RMG1 (SEQ ID NO: 12), RMB3 (SEQ ID NO: 18), RMC2 (SEQ ID NO: 13).

FIG. 5. Binding assessment via flow cytometric analysis of purified derivative anti-RBC RMA1 and RMC1 to human and mouse red blood cells. RMC1 (SEQ ID NO: 2) and RMA1 (SEQ ID NO: 9) are the affinity-matured antibodies to mouse RBC.

FIG. 6. (A) SDS-PAGE of purified anti-Trastuzumab-TPT and anti-RBC-TPT fusion bispecific antibody RMC1-TF7. (B) Binding activity of the fusion bispecific sdAbs against mouse RBC. (C) PK study with the fusion constructs in mouse. 1HE and TF7 are control non-binding antibodies. RMC1-TF7 and 1HE-TF7 are bispecific constructs.

FIG. 7. (A) SDS-PAGE of purified anti-RBC-DM4 fusion bispecific antibody RMC1-DMFH1 and RMC1-DMOH9. (B) Binding activity of the fusion bispecific sdAbs against free DM4 was evaluated via competitive ELISA. DMFH1 and DMOH9 are representative anti-maytansinoid antibodies.

FIG. 8. Assessment of simultaneous binding of the fusion anti-RBC-DM4 bispecific antibodies to both mouse RBC and DM4. Mouse RBCs were incubated with the fusion proteins, then biotin-DM4 or biotin-MMAF was added. Binding activity was detected with PE fluorophore conjugated strepavidin. TF7 is a control non-binding antibody.

FIG. 9. Comparison of the anti-DM4 antagonism activity of the fusion antibodies to their parent anti-DM4 sdAbs. In FIG. 9, for each concentration shown, the legend indicates the sample tested from left to right, starting with DM4 af the top of the legend.

### DETAILED DESCRIPTION OF THE DISCLOSURE

Where a range of values is provided in this disclosure, it should be understood that each intervening value, to the tenth of the value of the lower limit of that range, and any other intervening value and ranges in that stated range are encompassed within the disclosure, unless clearly indicated otherwise. The upper and lower limits of these smaller ranges may independently be included in the smaller ranges encompassed within the disclosure.

As used in this disclosure, the singular forms include the plural forms and vice versa unless the context clearly indicates otherwise. The indefinite articles “a” and “an” as used in the specification and claims should be understood to mean “at least one” unless clearly indicated otherwise.

A general reference to an “antibody” in this disclosure is also intended to include all full-length single domain antibodies, fragments thereof containing antigen binding domains, as well as modified antibodies or fragments containing substitutions or modifications of the amino acid residues, and including where antibody fragments, modified or not, may be linked together via covalent linkages with or without linkers.

The present disclosure provides compositions and methods using binding partners that bind with specificity to an erythrocyte protein, illustrated using SLC4A1 transporter (also referred to as Band 3) that is highly expressed on erythrocytes. Such binding partners as described above are referred to as EBPs in the plural and EBP in the singular. The binding partners may be provided as bi- or multi-specific agents that have improved half-life and/or are effective at a lower concentration. The disclosure thus provides an EBP as a first component of a multi-component molecule, and wherein a second component is a binding partner that specifically binds to another agent, such as a biologically active compound (i.e., payload) that is administered to an individual. The second component may therefore be referred to as a payload-binding selectivity enhancer (PBSE). In non-limiting embodiments the PBSE specifically binds to a drug that was administered to an individual as a component of an ADC. Accordingly, in embodiments, an ADC that may be used in conjunction with the described partners comprises an antibody group and a drug group. The antibody group and the drug group may be connected using any suitable linker. For any particular ADC the antibody group may be referred to as an antibody or by the name of the antibody. Similarly, the drug group may be referred to as a drug or by the name of the drug. In an embodiment, an ADC that is used with a described bi- or multi-specific construct comprises Trastuzumab. In a non-limiting embodiment the disclosure provides a bispecific construct that contains an EBP as a first component and a second component that specifically binds to Trastuzumab, such as the described RMC1-TF7 construct. Another non-limiting example is the 1HE-TF7 construct.

The first component of a described binding partner targets an antigen present on an erythrocyte, such as the SLC4A1 transporter. In embodiment a linking group may be used to attach the first and second components to one another to produce a bi-specific construct.

The term “treatment” as used herein refers to reduction or delay in one or more symptoms or features associated with the presence of the particular condition being treated Treatment does not necessarily mean complete cure and does not preclude relapse, but may be used in connection with any such relapse.

The term “therapeutically effective amount” as used herein is the amount sufficient to achieve, in a single or multiple doses, the intended purpose of treatment. The exact amount desired or required will vary depending on the mode of administration, patient specifics and the like. Appropriate effective amounts can be determined by one of ordinary skill in the art (such as a clinician) with the benefit of the present disclosure.

The EBP may be a whole single chain immunoglobulin or an antigen binding fragment thereof. The described antibodies or antigen binding fragments thereof may be used in a variety of antibody platforms, such as single-chain antibodies (scFv), bivalent antibodies, diabodies, and the like.

As discussed above, in embodiments, an EBP specifically binds to a red blood cell protein as described herein may be provided in a bispecific format, wherein the non-red blood cell binding component of the bispecific antibody specifically binds to a payload and thus comprises the PBSE. In this embodiment, the payload may also be administered to the individual. Thus, all description of drugs/payloads that may be conjugated to a described antibody to form an ADC, and used in conjunction with a described bi- or multispecific binding partner as described herein include those payloads for use in their non-conjugated form.

A described antibody may be a chimeric antibody. The term “chimeric antibody” refers to an antibody that has framework residues from one species, such as human, and CDRs (which generally confer antigen binding) from another species, such as camelid or shark antibody that specifically binds an erythrocyte protein. In a chimeric antibody, some portions may be identical or homologous to sequences from a particular species while other portions may be identical or homologous to sequences from a different species.

The described binding partners may be partially or fully humanized. A “human” antibody (also called a “fully human” antibody) is an antibody that includes human framework regions and all of the CDRs from a single or different human immunoglobulins. Thus, frameworks from one human antibody can be engineered to include CDRs from a different human antibody.

A “humanized antibody” is typically a human antibody that has one or more amino acid residues imported into it (i.e., introduced into it) from a source that is non-human. For example, a humanized antibody is a recombinant protein in which the CDRs of an antibody from a species such a camelid or shark. The constant domains (also referred to as framework regions) of the antibody molecule are generally the same as those of a human antibody. The non-human immunoglobulin providing the CDRs can be termed as “donor” and the human immunoglobulin providing the framework can be termed as “acceptor”. Constant regions need not be always present, but if they are, they can be substantially identical to human immunoglobulin constant regions, i.e., at least about 85-90%, such as about 95% or more identical.

The present disclosure provides sequences that have homology with the antibody sequences described herein. In various examples, the homologous sequences have at least 85%, 86%, 87%, 88%, 89%, 90%, 91%, 92%, 93%, 94%, 95%, 96%, 97%, 98%, or 99% identity with a protein or peptide sequence of the present disclosure.

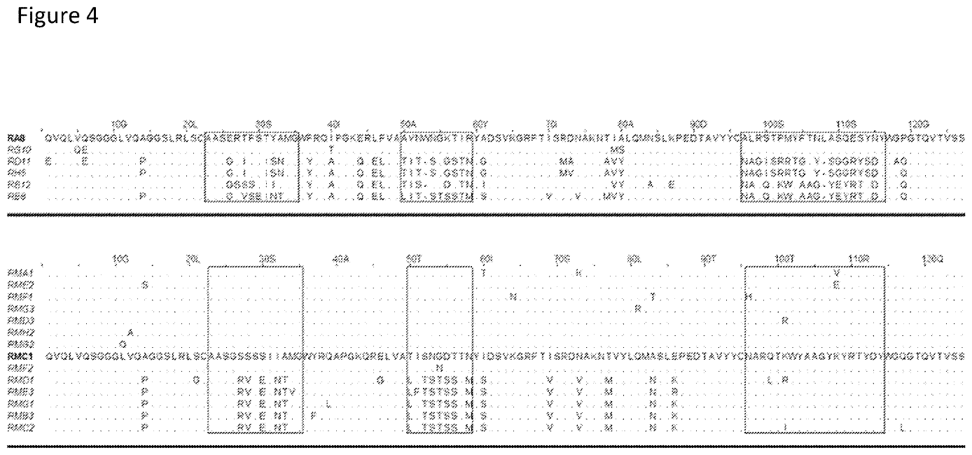

The disclosure includes single domain antibodies and antigen binding fragments thereof that comprise the CDRs as depicted in FIG. 4.

The payload molecule to which the PBSE component of a described binding partner may is typically a drug molecule that causes cell toxicity. In an embodiment, when the drug molecule is conjugated to an antibody in an ADC the drug molecule may be covalently bound to an N-terminal amino acid of the antibody via a reactive group or via a linker. Examples of reactive groups that cross link with the N-terminal alpha amine group of the antibody (such as light or heavy chain) include isothiocyanate, isocyate, acyl azide, NHS ester, sulfonyl ester, aldehyde, glyoxal, epoxide, carbonate, aryl halide, imidoester, carbodiimide, anhydride, fluorophenyl ester and the like. The reactive groups aldehyde or NHS ester are commonly used. Reduced cysteines (free sulfhydryls) may also be used. Conjugation methods of payload molecules to antibody are known. For example, conjugation of payload molecules to antibody is described in U.S. Pat. No. 10,071,170, the description of which is incorporated herein by reference.

Several antibodies (including fragments or modifications) are described in the examples herein, and sequences are disclosed. Generation of the antibodies, screening of the antibodies to identify the candidates with the desired binding specificity and affinity, and their binding and inhibitory characteristics are described.

The sequences of any of the antibodies or fragments described herein may include a polyhistidine tag, or the sequences may be used without the polyhistidine tag (e.g., to reduce immunogenicity). Any specific sequences disclosed here with polyhistidine tags also include the corresponding sequences without the polyhistidine tags, and any sequences disclosed herein without the polyhistidine tags also include sequences with the polyhistidine tags. Variants of the sequences of antibodies or fragments disclosed herein include sequences which have at least 85% identity with the disclosed sequences providing the binding affinity is not adversely affected. For example, the binding affinity of variants may be 10% less, the same, or better than the disclosed sequences. In embodiments, the variants may have at least 90%, at least 95%, at least 98%, at least 99% homology (identity) with the disclosed sequences without adversely affecting the binding affinity. The disclosure also includes nucleotide sequences that encode for the amino acid sequences or their variants as described herein.

In embodiments, a described binding partner forms a complex with a protein that is connected to an erythrocyte membrane.

In an aspect, the disclosure provides pharmaceutical compositions comprising or consisting essentially of the binding partners as described herein. The formulations typically contain physiologically acceptable carriers, excipients, or stabilizers and may be in the form of aqueous solutions, lyophilized or other dried or solid formulations. Examples of suitable pharmaceutical preparation components can be found in Remington: The Science and Practice of Pharmacy 22nd edition (2012). Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™, polyethylene glycol (PEG) and the like. In an embodiment, the pharmaceutical composition may comprise buffer components and stabilizers, including, but not limited to, sucrose, polysorbate 20, NaCl, KCl, sodium acetate, sodium phosphate, arginine, lysine, trehalose, glycerol, and maltose. In embodiments, the ADCs or fragments or modifications thereof are the only protein molecules present in the compositions.

Compositions comprising a described binding partner may be administered using any suitable route including parenteral, subcutaneous, intraperitoneal, intrapulmonary, and intranasal. Parenteral infusions include intramuscular, intravenous, intraarterial, intraperitoneal, or subcutaneous administration. The administration(s) may be carried out in a continuous manner or may be intermittent. Appropriate dosage will depend upon the particular tumor being treated, the specifics and condition of the individual patient, the mode of administration etc. Determination of appropriate dosage is within the purview of one skilled in the art, such as a treating physician.

The amount of described binding partner can be administered to an individual in need of treatment at a dose that is effective to treat a solid tumor. In general, suitable dosages of the antibodies or fragments thereof can range from about 0.1 mg/kg to 100 mg/kg, including all 0.1 mg/kg values and ranges there between. Examples of dosages include 0.1, 0.5, 1, 5, 10, 15, 20, 25, 30, 35, 40, 45, 50, 55, 60, 65, 70, 75, 80, 85, 90, 95 and 100 mg/kg. A variety of dosage regimens are contemplated including dosage regimens in which the ADC may be administered repeatedly, e.g., on a daily, weekly or monthly schedule, over a short period or an extended period of time, e.g., months to years (e.g., maintenance therapy).

In an embodiment, additional half-life extension strategies can be used to increase plasma half-life the described antibodies including PASylation and PEGylation.

The present composition/compositions may be administered alone or in combination with other types of treatments (e.g., surgical resection, radiation therapy, chemotherapy, hormonal therapy, immunotherapy, or other anti-tumor agents).

The present compositions may be used for any type of cancer, including carcinoma, lymphoma, sarcoma, melanoma, and leukemia, in settings where an anti-cancer payload is used. Non-limiting examples include squamous cell cancer, small-cell lung cancer, non-small cell lung cancer, adenocarcinoma of the lung, squamous carcinoma of the lung, cancer of the peritoneum, myeloma (including multiple myeloma), hepatocellular cancer, gastrointestinal cancer, pancreatic cancer, glioblastoma/glioma (e.g., anaplastic astrocytoma, glioblastoma multiforme, anaplastic oligodendroglioma, anaplastic oligodendroastrocytoma), cervical cancer, ovarian cancer, liver cancer, bladder cancer, hepatoma, breast cancer, brain cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

Examples of payload molecules to which a described bi- or other multi-valent binding partner can bind include microtubule formation inhibitors, meiosis inhibitors, topoisomerase inhibitors, RNA polymerase inhibitors, DNA intercalators or alkylators, ribosome inhibitors, enzymes (carboxypeptidase, alkaline phosphatase, cytosine deaminase), immunocytokines (e.g. Interleukin-2) and the like. Examples of cytotoxic drugs include, but are not limited to, maytansinoid, auristatin, dolastatin, tubulysin, camptothecin, pyrrolobenzodiazepines, calicheamicin, gelonin, doxorubicin, duocamysin, carboplatin, cisplatin, cyclophosphamide, ifosfamide, nidran, bleomycin, mitomycin C, cytarabin, fluorouracil, methotrexate, trimetrexate, vinblastine, alimta, altretamine, procarbazine, taxol, taxotere, diphtheria toxin, Pseudomonas exotoxin and derivatives (e.g. PE38, PE40), alpha-emitters (Ac-225, At-211, Th-227, Ra-223, Pb-212, Bi-212, Ra-224). Compounds include their stereoisomers and derivatives. Auristatin may be monomethyl auristatin E (MMAE) or monomethyl auristatin F (MMAF).

In embodiments the payload is cleaved from an ADC and bound by a described binding partner. In embodiments, a described binding partner only specifically binds to a free payload, or exhibits preferential binding to a free payload relative to the payload when present in an ADC. When the payload is cleaved from the ADC, such as via enzymatic action, hydrolysis, oxidation, or some other mechanism, the cleaved payload may comprise a portion of the linking group, all of the linking group, or none of the linking group. The cleaved payload may further comprise substituents that may be added during or after cleavage, or functional groups formed as a result of the cleavage process. Examples of substituents and/or functional groups formed from the cleavage process include, but are not limited to, alkyl groups (e.g., methyl, ethyl, propyl, isopropyl, and the like), alcohol groups, amines, thiols, sulfonic acids, sulfoxides, sulfides, sulfones, carboxylic acids, esters, amides, and the like, and combinations thereof.

In an embodiment, the present disclosure provides compositions and methods for treatment of cancer using one or more described binding partners, where the drug is camptothecin or a camptothecin derivative, analog, or metabolite. The camptothecin derivative, analog, or metabolite thereof can be topotecan. The camptothecin derivative, analog, or metabolite thereof can be irinotecan. The camptothecin derivative, analog, or metabolite thereof can be exatecan.

In an embodiment, the present disclosure provides compositions and methods for treatment of cancer using a described binding partner, where the drug is calicheamicin or a calicheamicin derivative, analog, or metabolite.

In an embodiment, the present disclosure provides compositions and methods for treatment of cancer using a described binding partner, where the drug is a maytansinoid or a maytansinoid derivative, analog or metabolite. The maytansinoid or derivative, analog, or metabolite thereof may be DM1, DM2, DM3, DM4, DM5, DM6, or DM7.

In embodiments the drug is an auristatin E (MMAE) or auristatin E derivative, analog, or metabolite. In an embodiment, the drug is a pyrrolobenzodiazepine (PBD) or PBD derivative, analog or metabolite.

The disclosure is further demonstrated by way of the figures and data presented herein, and the following Examples, which are not intended to be limiting.

### Example 1

### Methods

### RBC Band 3 Extraction

### Preparation of Ghost Membranes

Human RBC (Rockland Immunochemicals, Limerick PA) were washed 2 times with 10 volumes of PBS Ph 7.4 and centrifuged at 3000 g for 5 min. The washed cells were mixed with 10 volumes of ice-cold 5P8 buffer (5 Mm sodium phosphate, Ph 8.0) containing 0.2 Mm DTT, 20 μg/ml PMSF, and incubated for 10 min on ice. The cell lysate was centrifuged for 20 min at 27,000 g at 4° C., and hemolysate was removed by aspiration. The ghost cell pellet was washed for additional 4 times.

### Stripping of Peripheral Proteins and Solubilization of Stripped Ghosts

The ghost cell membrane was washed with 10 vol of ice-cold SE buffer, then centrifuged at 46,000 g for 20 min at 4° C. The pellet was re-suspended with a syringe in 10 vol of SE buffer, incubated at 37° C. for 30 min, then centrifuged at 46,000 g for 20 min at 4° C. This process was repeated one more time before re-suspending the spectrin-depleted ghost membrane with a syringe in 10 vol of KI extraction buffer, and incubated for 30 min at 37° C. The ghost membrane was pelleted by centrifugation for 30 min at 46,000 g at 4° C., then re-suspended in 5P8+PMSF buffer, and incubated at 4° C. for 10 min. The ghost membrane was centrifuged at 46,000 g for 30 min, and washed once more time with 5P8+PMSF buffer. To solubilize the KI-extracted ghosts, 4 vol of 1% C₁₂E₈ (v/v) in 228 Mm sodium citrate Ph 8.0+1 Mm DTT+PMSF was added, and incubated for 20 minutes on ice. The resulting suspension was centrifuged at 46,000 g for 30 min (or up to 80,000 g for 45 min) at 4° C., and the supernatant was collected and stored at 4° C.

### Llama Immunization and Phage Display Construction

To establish anti-RBC sdAb phage display library, a llama provided by Capralogics (Hardwick, MA) was subcutaneously immunized with 300 μg of extracted RBC Band 3 in complete Freund's adjuvant every three weeks for a total of four immunizations. Ten days after the fourth immunization, 600 Ml of freshly harvested blood was used to isolate PBMCs, and phage display library was constructed by using the method described previously.

### Example 2

### Results

### RBC Immunization, Panning and Screening

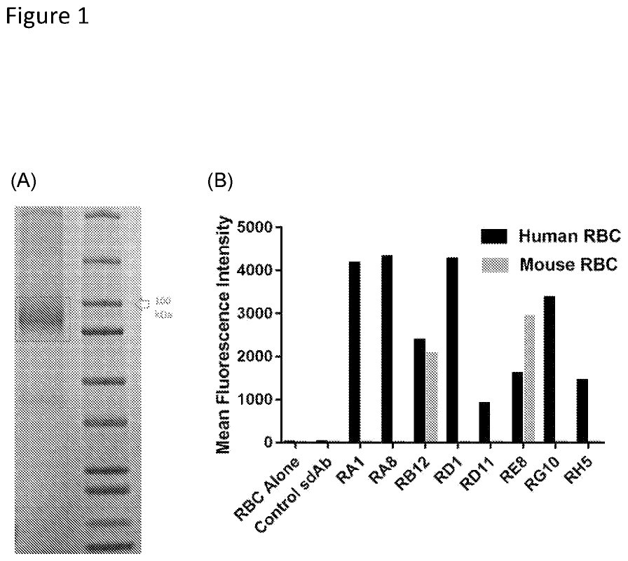

In preparation for llama immunization, bio-panning, and screening, RBC Band 3 antigen was extracted from human RBC. SDS-PAGE gel analysis of the extracted band 3 from human RBC showed a prominent band of the band 3 antigen at around 95 kDa with an acceptable purity (FIG. 1A). Each 10 Ml of 10% human RBC commercial solution yields around 500 μg of extracted band 3 protein. A sdAb phage display library was built from PBMCs of a llama immunized with the extracted band 3 antigen, panned against human RBC, and screened with ELISA and flow cytometry. As shown in FIG. 1B, eight positive sdAb clones binding to human RBC were identified, and among them, only RE8 (SEQ ID NO: 6) and RB12 (SEQ ID NO: 1) clones showed binding activity to mouse RBC. In this regard, one aspect of the disclosure pertains to bi- and multi-specific antibody formats for therapeutic uses in humans, and therefore selectively bind to human erythrocites. In some embodiments the described anti-human-RBC antibodies can cross-react with mouse RBC, such as RE8 (SEQ ID NO: 6) and RB12 (SEQ ID NO: 1), are accordingly suitable for animal model research purposes and may also be useful as human therapeutics.

Characterizations of the Positive Anti-RBC sdAbs

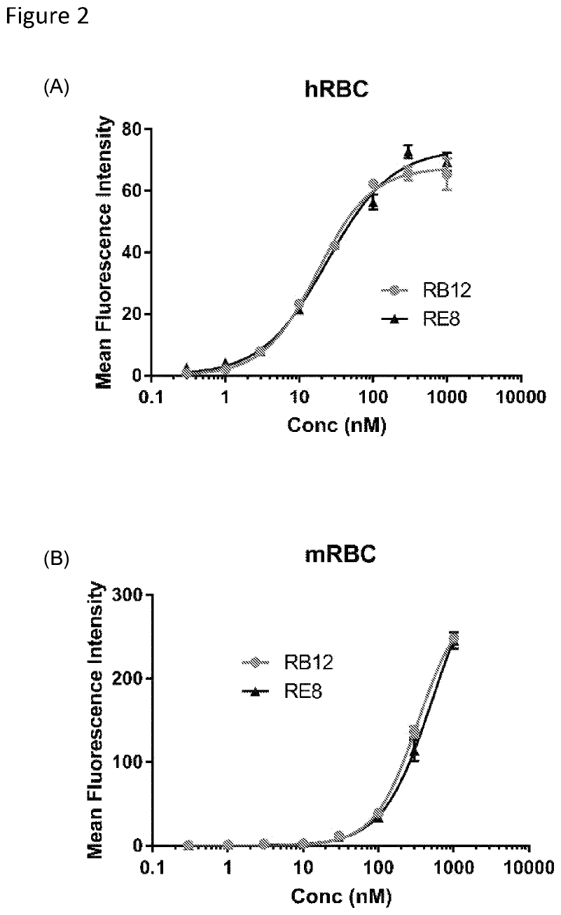

Binding affinity of the two anti-RBC sdAb clones that showed binding to both human and mouse RBC were determined via flow cytometry. Both clones showed high binding affinity to human RBC with the estimated KD of 17.7 Nm and 23.6 Nm for RB12 (SEQ ID NO: 1), and RE8 (SEQ ID NO: 6), respectively (FIG. 2A). The binding affinity of these clones to mouse RBC revealed to be lower than to human RBC, with the estimated KD of 335 Nm and 528 Nm for RB12 (SEQ ID NO: 1), and RE8 (SEQ ID NO: 6), respectively (FIG. 2B).

Pharmacokinetics of Anti-RBC sdAbs in Mice

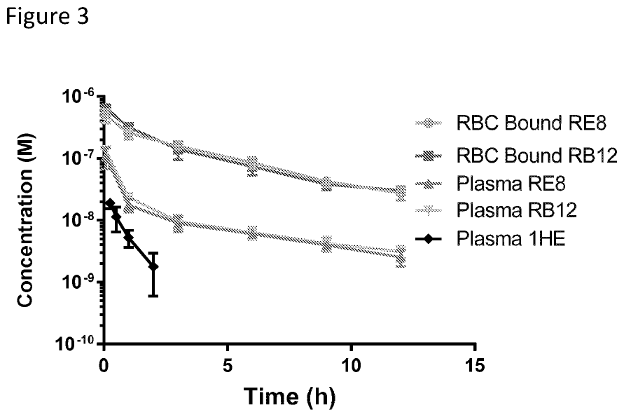

The concentration time profile after an IV administration of 1 mg/kg of RB12 and RE8 with tracer dose of 1125 labeled is shown in FIG. 3. RBC and plasma were separated from whole blood, TCA precipitated and radioactivity counted. Plasma half-life improved from minutes for a non-binding sdAb to 2.5 hours for the two RBC-binding sdAbs RE8 (SEQ ID NO: 6) and RB12 (SEQ ID NO: 1) that has moderate to low affinities to mouse RBC (KD of 300 Nm)

### Affinity Maturation of RB12 (SEQ ID NO: 1) and RE8 (SEQ ID NO: 6)

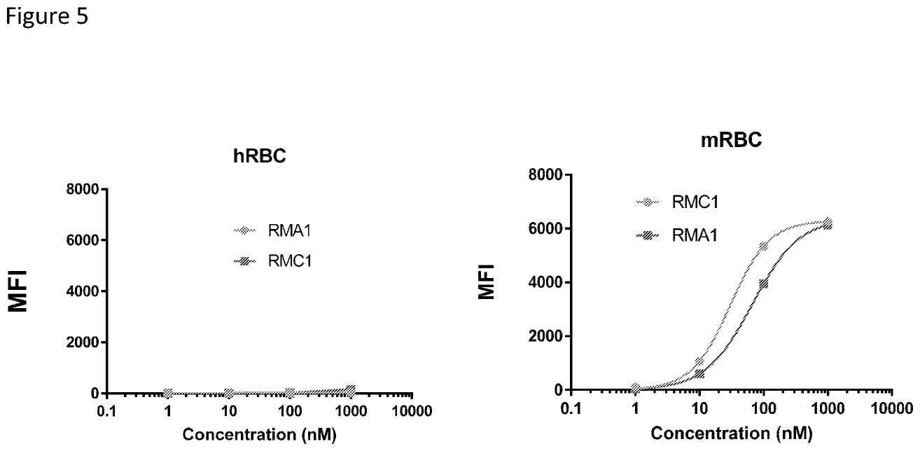

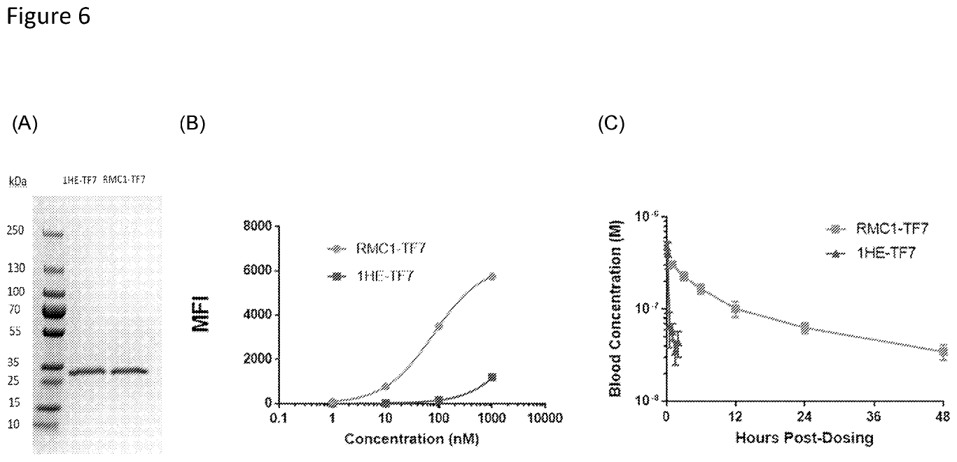

A derivative library was constructed from RB12 (SEQ ID NO: 1) and RE8 (SEQ ID NO: 6) via random mutagenesis, and affinity maturation was performed through additional panning and screening procedures to isolate clones with higher affinity to mouse RBC. Several derivative clones were identified, and majority of the mutated clones have a common single point mutation at E109 to Lysine, with an exception of RMA1 (SEQ ID NO: 9) (to Valine instead) (FIG. 4). Further binding assessment of the 2 clones RMA1 (SEQ ID NO: 9) and RMC1 (SEQ ID NO: 2) was carried out via flow cytometry. There is no binding detected to human RBC up to 1 μM of purified sdAbs, while improved binding affinity to mouse RBC was observed with the estimated KD for RMA1 (SEQ ID NO: 9) and RMC1 (SEQ ID NO: 2) are 66.9 Nm and 30.3 Nm respectively (FIG. 5). RMC1-TF7 (RBC-binding anti-SN38 PBSE) and 1HE-TF7 (RBC-nonbinding PBSE control) were constructed (FIG. 6A). The estimated KD of RMC1-TF7 is 70 Nm, while 1HE-TF7 has some degree of nonspecific binding at very high concentration of 1 Mm (FIG. 6B). RBC binding PBSE RMC1-TF7 showed significant longer circulation half-life (around 10 hours) than nonbinding PBSE 1HE-TF7 (around 30 minutes) (FIG. 6C).

### Development and Assessment of Anti-RBC-DM4 Fusion Antibodies

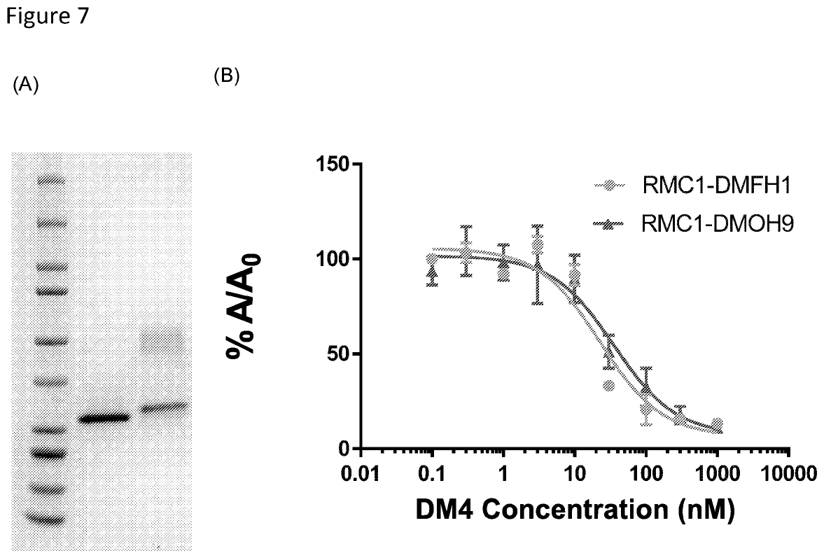

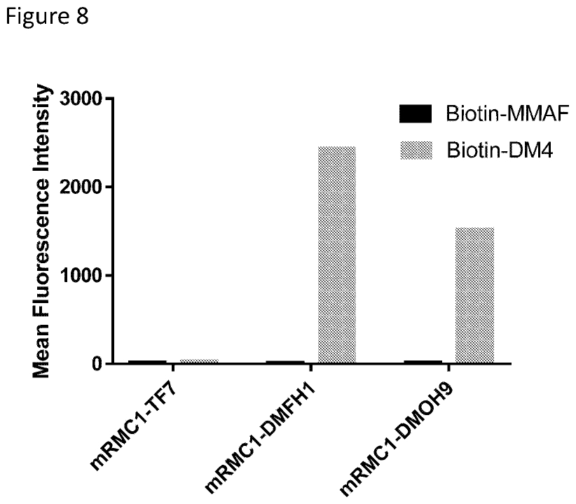

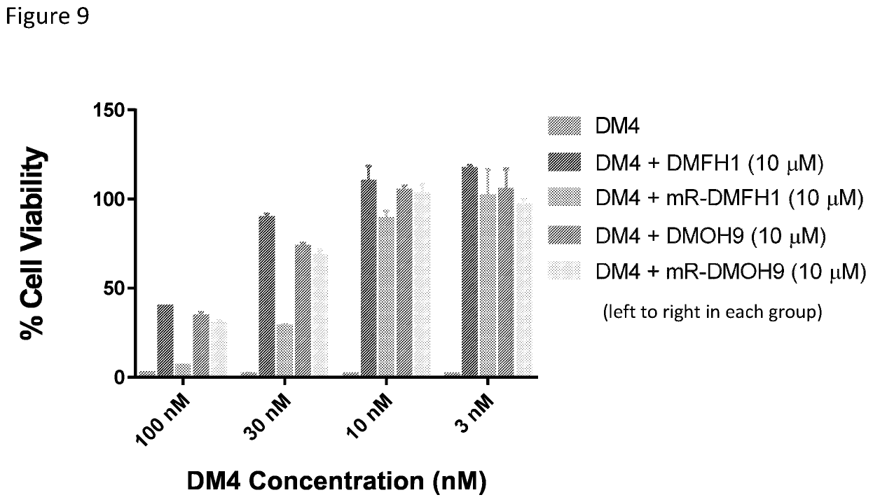

Anti-RBC-DM4 bispecific fusion constructs was developed and characterized to evaluate binding activity from the lead anti-DM4 sdAbs with the highest antagonism activity to DM4, DMFH1 and DMOH9, with RMC1 (SEQ ID NO: 2). The constructs have sizes around 33 kDa (FIG. 7A), and their binding affinity KD to free DM4 are estimated via competitive ELISA to be 22 Nm and 35 Nm for RMC1-DMFH1 and RMC1-DMOH9, respectively (FIG. 7B). Simultaneous binding to both RBC and DM4 was assessed via flow cytometry. As shown in FIG. 8, the constructs were able to bind to both mouse RBC and DM4-biotin concurrently. Antagonism activity of these constructs to DM4 cytotoxicity was determined in SK-BR-3 cells. RMC1-DMOH9 have similar degree of protection from DM4 cytotoxicity to its parent sdAb DMOH9 across all tested concentrations, whereas RMC1-DMFH1 exhibited a reduction in activity at higher concentration of 100 Nm and 30 Nm DM4 (FIG. 9).

Representative antibody sequences generated and characterized as described above are as follows wherein the antibody sequences are assigned alphanumeric identifiers:

>RB12
(SEQ ID NO: 1)
QVQLVQSGGGLVQAGGSLRLSCAASGSSSSIIAMGWYRQAPGKQRELVATISNGD
TTNYIDSVKGRFTISRDNAKNTVYLQMASLEPEDTAVYYCNARQTKWYAAGYEYRT
YDYWGQGTQVTVSS
>RMC1
(SEQ ID NO: 2)
QVQLVQSGGGLVQAGGSLRLSCAASGSSSSIIAMGWYRQAPGKORELVATISNGD
TTNYIDSVKGRFTISRDNAKNTVYLQMASLEPEDTAVYYCNARQTKWYAAGYKYRT
YDYWGQGTQVTVSS
>RA8
(SEQ ID NO: 3)
QVQLVQSGGGLVQAGGSLRLSCAASERTFSTYAMGWFRQIPGKERLFVAAVNWN
GKTIRYADSVKGRFTISRDNAKNTIALQMNSLKPEDTAVYYCALRSTPMYFTNLASQE
SYNYWGPGTQVTVSS
>RD1
(SEQ ID NO: 4)
QVQLVQSGGGLVQAGGSLRLSCAASGSSSSIIAMGWYRQAPGKQRELVATISNGD
TTNYIDSVKGRFTISRDNAKNTVYLQMASLEPEDTAVYYCNARQTKWYAAGYEYRT
YDYWGQGTQVTVSS
>RD11
(SEQ ID NO: 5)
EVQLVESGGGLVQPGGSLRLSCAASGRIFSISNMGWYRQAPGKQRELVATITSGGS
TNYGDSVKGRFTISMANAKNAVYLQMNSLKPEDTAVYYCNAGISRRTGTYSGGRYS
DYWAQGTQVTVSS
>RE8
(SEQ ID NO: 6)
QVQLVQSGGGLVQPGGSLRLSCAASGRVSEINTMGWYRQAPGKQRELVALITSTS
STMYSDSVKGRFTVSRDVAKNMVYLQMNSLKPEDTAVYYCNARQTKWYAAGYEY
RTYDYWGQGTQVTVSS
>RG10
(SEQ ID NO: 7)
QVQLQESGGGLVQAGGSLRLSCAASERTFSTYAMGWFRQTPGKERLFVAAVNWN
GKTIRYADSVKGRFTISRDNAKNTMSLQMNSLKPEDTAVYYCALRSTPMYFTNLASQ
ESYNYWGPGTQVTVSS
>RH5
(SEQ ID NO: 8)
QVQLVQSGGGLVQPGGSLRLSCAASGRIFSISNMGWYRQAPGKQRELVATITSGG
STNYGDSVKGRFTISMVNAKNAVYLQMNSLKPEDTAVYYCNAGISRRTGTYSGGRYS
DYWGQGTQVTVSS
>RMAI
(SEQ ID NO: 9)
QVQLVQSGGGLVQAGGSLRLSCAASGSSSSIIAMGWYRQAPGKQRELVATISNGD
TTNYTDSVKGRFTISRDKAKNTVYLQMASLEPEDTAVYYCNARQTKWYAAGYVYR
TYDYWGQGTQVTVSS
>RMDI
(SEQ ID NO: 10)
QVQLVQSGGGLVQPGGSLRLGCAASGRVSEINTMGWYRQAPGKQRGLVALITST
SSTMYSDSVKGRFTVSRDVAKNMVYLQMNSLKPEDTAVYYCNARLTRWYAAGYKY
RTYDYWGQGTQVTVSS
>RMF1
(SEQ ID NO: 11)
QVQLVQSGGGLVQAGGSLRLSCAASGSSSSIIAMGWYRQAPGKQRELVATISNGD
TTNYIDSVNGRFTISRDNAKNTVYLQMTSLEPEDTAVYYCHARQTKWYAAGYKYRT
YDYWGQGTQVTVSS
>RMGI
(SEQ ID NO: 12)
QVQLVQSGGGLVQPGGSLRLSCAASGRVSEINTMGWYRLAPGKQRELVALITSTS
STMYSDSVKGRFTVSRDVAKNMVYLQMNSLKPEDTAVYYCNARQTKWYAAGYKY
RTYDYWGQGTQVTVSS
>RMC2
(SEQ ID NO: 13)
QVQLVQSGGGLVQPGGSLRLSCAASGRVSEINTMGWYRQAPGKQRELVALITSTS
STMYSDSVKGRFTVSRDVAKNMVYLQMNSLKPEDTAVYYCNARQTIWYAAGYKYR
TYDYWGLGTQVTVSS
>RME2
(SEQ ID NO: 14)
QVQLVQSGGGLVQSGGSLRLSCAASGSSSSIIAMGWYRQAPGKQRELVATISNGD
TTNYIDSVKGRFTISRDNAKNTVYLQMASLEPEDTAVYYCNARQTKWYAAGYEYRT
YDYWGQGTQVTVSS
>RMF2
(SEQ ID NO: 15)
QVQLVQSGGGLVQAGGSLRLSCAASGSSSSIIAMGWYRQAPGKQRELVATISNND
TTNYIDSVKGRFTISRDNAKNTVYLQMASLEPEDTAVYYCNARQTKWYAAGYKYRT
YDYWGQGTQVTVSS
>RMG2
(SEQ ID NO: 16)
QVQLVQSGGGQVQAGGSLRLSCAASGSSSSIIAMGWYRQAPGKQRELVATISNGD
TTNYIDSVKGRFTISRDNAKNTVYLQMASLEPEDTAVYYCNARQTKWYAAGYKYRT
YDYWGQGTQVTVSS
>RMH2
(SEQ ID NO: 17)
QVQLVQSGGGLAQAGGSLRLSCAASGSSSSIIAMGWYRQAPGKQRELVATISNGD
TTNYIDSVKGRFTISRDNAKNTVYLQMASLEPEDTAVYYCNARQTKWYAAGYKYRT
YDYWGQGTQVTVSS
>RMB3
(SEQ ID NO: 18)
QVQLVQSGGGLVQPGGSLRLSCAASGRVSEINTMGWFRQAPGKQRELVALITSTS
STMYSDSVKGRFTVSRDVAKNMVYLQMNSLKPEDTAVYYCNARQTKWYAAGYKY
RTYDYWGQGTQVTVSS
>RMD3
(SEQ ID NO: 19)
QVQLVQSGGGLVQAGGSLRLSCAASGSSSSIIAMGWYRQAPGKQRELVATISNGD
TTNYIDSVKGRFTISRDNAKNTVYLQMASLEPEDTAVYYCNARQTRWYAAGYKYRT
YDYWGQGTQVTVSS
>RME3
(SEQ ID NO: 20)
QVQLVQSGGGLVQPGGSLRLSCAASGRVSEINTVGWYRQAPGKQRELVALFTSTS
STMYSDSVKGRFTVSRDVAKNMVYLQMNSLRPEDTAVYYCNARQTKWYAAGYKY
RTYDYWGQGTQVTVSS
>RMG3
(SEQ ID NO: 21)
QVQLVQSGGGLVQAGGSLRLSCAASGSSSSIIAMGWYRQAPGKQRELVATISNGD
TTNYIDSVKGRFTISRDNAKNTVYLRMASLEPEDTAVYYCNARQTKWYAAGYKYRT
YDYWGQGTQVTVSS

While the invention has been described through illustrative examples, routine modifications will be apparent to those skilled in the art, which modifications are intended to be within the scope of the present disclosure.

1. A single chain binding partner that specifically binds to SLC4A1 transporter/Band 3, wherein the single chain binding partner comprises or consists of a sequence selected from:
>RB12
(SEQ ID NO: 1)
QVQLVQSGGGLVQAGGSLRLSCAASGSSSSIIAMGWYRQAPGKQRELVATISNGDTT
NYIDSVKGRFTISRDNAKNTVYLQMASLEPEDTAVYYCNARQTKWYAAGYEYRTYDY
WGQGTQVTVSS
>RMC1
(SEQ ID NO: 2)
QVQLVQSGGGLVQAGGSLRLSCAASGSSSSIIAMGWYRQAPGKQRELVATISNGDTT
NYIDSVKGRFTISRDNAKNTVYLQMASLEPEDTAVYYCNARQTKWYAAGYKYRTYDY
WGQGTQVTVSS
>RA8
(SEQ ID NO: 3)
QVQLVQSGGGLVQAGGSLRLSCAASERTFSTYAMGWFRQIPGKERLFVAAVNWNGK
TIRYADSVKGRFTISRDNAKNTIALQMNSLKPEDTAVYYCALRSTPMYFTNLASQE
SYNYWGPGTQVTVSS
>RD1
(SEQ ID NO: 4)
QVQLVQSGGGLVQAGGSLRLSCAASGSSSSIIAMGWYRQAPGKQRELVATISNGDTT
NYIDSVKGRFTISRDNAKNTVYLQMASLEPEDTAVYYCNARQTKWYAAGYEYRTYDY
WGQGTQVTVSS
>RD11
(SEQ ID NO: 5)
EVQLVESGGGLVQPGGSLRLSCAASGRIFSISNMGWYRQAPGKQRELVATITSGGSTN
YGDSVKGRFTISMANAKNAVYLQMNSLKPEDTAVYYCNAGISRRTGTYSGGRYSDYWA
QGTQVTVSS
>RE8
(SEQ ID NO: 6)
QVQLVQSGGGLVQPGGSLRLSCAASGRVSEINTMGWYRQAPGKQRELVALITSTSST
MYSDSVKGRFTVSRDVAKNMVYLQMNSLKPEDTAVYYCNARQTKWYAAGYEYRTYD
YWGQGTQVTVSS
>RG10
(SEQ ID NO: 7)
QVQLQESGGGLVQAGGSLRLSCAASERTFSTYAMGWFRQTPGKERLFVAAVNWNG
KTIRYADSVKGRFTISRDNAKNTMSLQMNSLKPEDTAVYYCALRSTPMYFTNLASQESY
NYWGPGTQVTVSS
>RH5
(SEQ ID NO: 8)
QVQLVQSGGGLVQPGGSLRLSCAASGRIFSISNMGWYRQAPGKQRELVATITSGGSTN
YGDSVKGRFTISMVNAKNAVYLQMNSLKPEDTAVYYCNAGISRRTGTYSGGRYSDYWG
QGTQVTVSS
>RMAI
(SEQ ID NO: 9)
QVQLVQSGGGLVQAGGSLRLSCAASGSSSSIIAMGWYRQAPGKQRELVATISNGDTT
NYTDSVKGRFTISRDKAKNTVYLQMASLEPEDTAVYYCNARQTKWYAAGYVYRTYDY
WGQGTQVTVSS
>RMDI
(SEQ ID NO: 10)
QVQLVQSGGGLVQPGGSLRLGCAASGRVSEINTMGWYRQAPGKQRGLVALITSTSST
MYSDSVKGRFTVSRDVAKNMVYLQMNSLKPEDTAVYYCNARLTRWYAAGYKYRTYD
YWGQGTQVTVSS
>RMF1
(SEQ ID NO: 11)
QVQLVQSGGGLVQAGGSLRLSCAASGSSSSIIAMGWYRQAPGKQRELVATISNGDTT
NYIDSVNGRFTISRDNAKNTVYLQMTSLEPEDTAVYYCHARQTKWYAAGYKYRTYDY
WGQGTQVTVSS
>RMG1
(SEQ ID NO: 12)
QVQLVQSGGGLVQPGGSLRLSCAASGRVSEINTMGWYRLAPGKQRELVALITSTSST
MYSDSVKGRFTVSRDVAKNMVYLQMNSLKPEDTAVYYCNARQTKWYAAGYKYRTYD
YWGQGTQVTVSS
>RMC2
(SEQ ID NO: 13)
QVQLVQSGGGLVQPGGSLRLSCAASGRVSEINTMGWYRQAPGKQRELVALITSTSST
MYSDSVKGRFTVSRDVAKNMVYLQMNSLKPEDTAVYYCNARQTIWYAAGYKYRTYDY
WGLGTQVTVSS
>RME2
(SEQ ID NO: 14)
QVQLVQSGGGLVQSGGSLRLSCAASGSSSSIIAMGWYRQAPGKQRELVATISNGDTT
NYIDSVKGRFTISRDNAKNTVYLQMASLEPEDTAVYYCNARQTKWYAAGYEYRTYDY
WGQGTQVTVSS
>RMF2
(SEQ ID NO: 15)
QVQLVQSGGGLVQAGGSLRLSCAASGSSSSIIAMGWYRQAPGKQRELVATISNNDTT
NYIDSVKGRFTISRDNAKNTVYLQMASLEPEDTAVYYCNARQTKWYAAGYKYRTYDY
WGQGTQVTVSS
>RMG2
(SEQ ID NO: 16)
QVQLVQSGGGQVQAGGSLRLSCAASGSSSSIIAMGWYRQAPGKQRELVATISNGDTT
NYIDSVKGRFTISRDNAKNTVYLQMASLEPEDTAVYYCNARQTKWYAAGYKYRTYDY
WGQGTQVTVSS
>RMH2
(SEQ ID NO: 17)
QVQLVQSGGGLAQAGGSLRLSCAASGSSSSIIAMGWYRQAPGKQRELVATISNGDTT
NYIDSVKGRFTISRDNAKNTVYLQMASLEPEDTAVYYCNARQTKWYAAGYKYRTYDY
WGQGTQVTVSS
>RMB3
(SEQ ID NO: 18)
QVQLVQSGGGLVQPGGSLRLSCAASGRVSEINTMGWFRQAPGKQRELVALITSTSST
MYSDSVKGRFTVSRDVAKNMVYLQMNSLKPEDTAVYYCNARQTKWYAAGYKYRTYD
YWGQGTQVTVSS
>RMD3
(SEQ ID NO: 19)
QVQLVQSGGGLVQAGGSLRLSCAASGSSSSIIAMGWYRQAPGKQRELVATISNGDTT
NYIDSVKGRFTISRDNAKNTVYLQMASLEPEDTAVYYCNARQTRWYAAGYKYRTYDY
WGQGTQVTVSS
>RME3
(SEQ ID NO: 20)
QVQLVQSGGGLVQPGGSLRLSCAASGRVSEINTVGWYRQAPGKORELVALFTSTSST
MYSDSVKGRFTVSRDVAKNMVYLQMNSLRPEDTAVYYCNARQTKWYAAGYKYRTYD
YWGQGTQVTVSS
and
>RMG3
(SEQ ID NO: 21)
QVQLVQSGGGLVQAGGSLRLSCAASGSSSSIIAMGWYRQAPGKQRELVATISNGDTT
NYIDSVKGRFTISRDNAKNTVYLRMASLEPEDTAVYYCNARQTKWYAAGYKYRTYDY
WGQGTQVTVSS

2. The single chain binding partner of claim 1, wherein the single chain binding partner comprises or consists of a sequence selected from:
>RB12
(SEQ ID NO: 1)
QVQLVQSGGGLVQAGGSLRLSCAASGSSSSIIAMGWYRQAPGKQRELVATISNGDTT
NYIDSVKGRFTISRDNAKNTVYLQMASLEPEDTAVYYCNARQTKWYAAGYEYRTYDY
WGQGTQVTVSS
>RMC1
(SEQ ID NO: 2)
QVQLVQSGGGLVQAGGSLRLSCAASGSSSSIIAMGWYRQAPGKQRELVATISNGDTT
NYIDSVKGRFTISRDNAKNTVYLQMASLEPEDTAVYYCNARQTKWYAAGYKYRTYDY
WGQGTQVTVSS
>RA8
(SEQ ID NO: 3)
QVQLVQSGGGLVQAGGSLRLSCAASERTFSTYAMGWFRQIPGKERLFVAAVNWNGK
TIRYADSVKGRFTISRDNAKNTIALQMNSLKPEDTAVYYCALRSTPMYFTNLASQE
SYNYWGPGTQVTVSS
>RD1
(SEQ ID NO: 4)
QVQLVQSGGGLVQAGGSLRLSCAASGSSSSIIAMGWYRQAPGKQRELVATISNGDTT
NYIDSVKGRFTISRDNAKNTVYLQMASLEPEDTAVYYCNARQTKWYAAGYEYRTYDY
WGQGTQVTVSS
>RD11
(SEQ ID NO: 5)
EVQLVESGGGLVQPGGSLRLSCAASGRIFSISNMGWYRQAPGKQRELVATITSGGSTN
YGDSVKGRFTISMANAKNAVYLQMNSLKPEDTAVYYCNAGISRRTGTYSGGRYSDYWA
QGTQVTVSS
>RE8
(SEQ ID NO: 6)
QVQLVQSGGGLVQPGGSLRLSCAASGRVSEINTMGWYRQAPGKQRELVALITSTSST
MYSDSVKGRFTVSRDVAKNMVYLQMNSLKPEDTAVYYCNARQTKWYAAGYEYRTYD
YWGQGTQVTVSS
>RG10
(SEQ ID NO: 7)
QVQLQESGGGLVQAGGSLRLSCAASERTFSTYAMGWFRQTPGKERLFVAAVNWNG
KTIRYADSVKGRFTISRDNAKNTMSLQMNSLKPEDTAVYYCALRSTPMYFTNLASQESY
NYWGPGTQVTVSS
and
>RH5
(SEQ ID NO: 8)
QVQLVQSGGGLVQPGGSLRLSCAASGRIFSISNMGWYRQAPGKQRELVATITSGGSTN
YGDSVKGRFTISMVNAKNAVYLQMNSLKPEDTAVYYCNAGISRRTGTYSGGRYSDYWG
QGTQVTVSS

3. The single chain binding partner of claim 2, wherein the wherein the single chain binding partner comprises or consists of the sequence of:
(RB12)
(SEQ ID NO: 1)
QVQLVQSGGGLVQAGGSLRLSCAASGSSSSIIAMGWYRQAPGKQR
ELVATISNGDTTNYIDSVKGRFTISRDNAKNTVYLQMASLEPEDT
AVYYCNARQTKWYAAGYEYRTYDYWGQGTQVTVSS

4. The single chain binding partner of claim 1, wherein the single chain binding partner is a component of a multi-specific antibody, and wherein the multi-specific binding partner includes a component that specifically binds to a drug.

5. The single chain binding partner of claim 4, wherein the drug is used as a component of an antibody-drug conjugate (ADC), and wherein the antibody component of the ADC comprises Trastuzumab.

6. The single chain binding partner of claim 4, wherein the drug is DM4.

7. The single chain binding partner of claim 5, wherein the drug is DM4.

8. A method comprising administering to an individual in need thereof a composition comprising a single chain binding partner of claim 1.

9. The method of claim 8, wherein the single chain binding partner is a component of a multi-specific antibody, wherein the multi-specific antibody includes a component that specifically binds to a drug, the method comprising administering the single chain antibody binding partner to an individual who has received an antibody-drug conjugate (ADC), and wherein a component of the multi-specific antibody specifically binds to the drug after the drug has been liberated from the ADC.

10. The method of claim 9, wherein the drug is DM4.

11. The method of claim 10, wherein the single chain binding comprises or consists of the sequence:
(RB12)
(SEQ ID NO: 1)
QVQLVQSGGGLVQAGGSLRLSCAASGSSSSIIAMGWYRQAPGKQR
ELVATISNGDTTNYIDSVKGRFTISRDNAKNTVYLQMASLEPEDT
AVYYCNARQTKWYAAGYEYRTYDYWGQGTQVTVSS

12. The method of claim 11, wherein the antibody component of the ADC comprises Trastuzumab.
